(19) United States
(12) Patent Application Publication
(10) Pub. No.: US 2006/0188899 A1
(43) Pub. Date: Aug. 24, 2006

(54) High speed DNA sequencer and method

(76) Inventor: N. Binz DeWalch, Houston, TX (US)

Correspondence Address: Dewalch Technologies, Inc., 6850 Wynnwood, Houston, TX, 77008, US

(21) Appl. No.: 11/244,550

(22) Filed: Oct. 6, 2005

Related U.S. Application Data
(60) Provisional application No. 60/616,955, filed on Oct. 7, 2004.

(51) Int. Cl. C12Q 1/68

(52) U.S. Cl. 435/6

(57) ABSTRACT

This invention relates to a device for the determination of the sequence of nucleic acids and other polymeric or chain type molecules. Specifically, the device analyzes a sample prepared by incorporating fluorescent tags at the end of copies of varying lengths of the sample to be sequenced. The sample is then vaporized, charged and accelerated down an evacuated chamber. The individual molecules of the sample are accelerated to different velocities because of their different masses, which cause the molecules to be sorted by length as they travel down the evacuated chamber. Once sorted, the stream of molecules is illuminated causing the fluorescent tags to emit light that is picked up by a detector. The output of the detector is then processed by a computer to yield of the sequence of the sample under analysis. The present invention improves over the prior art by using photo-detection of the individual molecules instead of measuring the time of flight to a detector that measure collisions. Unlike mass spectrometry, the method of the present invention does not require the extreme sensitivity required to differentiate between very small mass differences in large molecules. The present invention is therefore more robust than the prior art and well suited for extremely high throughput sequencing of large nucleic acid molecules.

### RELATED CASES

The instant application claims priority to prior provisional application No. 60/616,955, filed Oct. 7, 2004.

### FIELD OF THE INVENTION

The present invention relates in general to the sequencing of DNA and other polymeric or chain type molecules and specifically to an apparatus and method that is capable of very high speed and throughput.

### BACKGROUND OF THE INVENTION

Current advances in the understanding of molecular biology and genetics as well as projects such as the Human Genome Project have created a growing demand for the DNA sequence of a multitude of organisms. The benefits to mankind in medicine, agriculture and for the environment as well as the economic potential that these fields promise are driving research to decipher the function of individual genes.

The amount of DNA sequence that organisms have varies from species to species but in all but the simplest organisms, the amount that must be determined is enormous. The Human Genome for example, consists of more than 3 billion bases that must be determined. The real benefit from genomics will not be derived from just the sequence data, it will be from an understanding of the function of the genes and the proteins that they encode. In order to determine the function and significance of different genes it is particularly helpful to compare the DNA sequence of entirely different species as well as the DNA sequence of like species. The DNA sequence varies even for organisms of the same species and it is these differences that determine the different characteristics of different individuals. By obtaining the sequence data from many different organisms and individuals and correlating the different characteristics with differences in the genes, great insight can be gained about genetic function. However, this requires very large amounts of sequencing capacity. There have been many methods and machines developed to improve the speed and throughput of DNA sequencing, however it has taken thousands of people, hundreds of machines and several years just to sequence the human genome using the current technology. This is entirely too slow and too costly to be practical to meet the future needs of genomics.

In order to provide background information so that the invention may be completely understood and appreciated in its proper context, reference is made to a number of prior art patents and publications as follows:

Currently there are two different sequencing approaches in use. The first method involves the use of electrophoresis and the second method involves the use of mass spectrography. The most common method in use involves the use of electrophoresis.

The general method of sequencing using electrophoresis involves the following steps:
a) Generation of multiple copies of different lengths of the segment of DNA to be sequenced using the polymerase chain reaction (PCR). During this reaction, a dideoxynucleoside triphosphate with a fluorescent tag molecule that corresponds to the original nucleotide is incorporated and terminates extension of the copy
b) Sorting the copies by length using gel electrophoresis
c) Determining the code after electrophoresis by individually illuminating the sorted molecules groups and determining the base at the end of the copy from the wavelength of light emitted by the particular fluorescent tag

U.S. Pat. No. 5,171,534 Smith et al. discloses a system for nucleic acid sequencing method that uses electrophoresis to sort by size, nucleic acid fragments prepared in a sequencing reaction. Each copy has a fluorescent tag that is substituted for the corresponding base. A laser illuminates the copies as they exit the electrophoresis medium and the base is determined by the color detected. This method of sequencing is widely used. The problem is that it relies on electrophoresis to sort the nucleic acid fragments, which is slow. Sorting of copies of DNA to sequence a segment having 1000 bases even in some of the fastest equipment can take up to an hour. Then the gel or medium for electrophoresis must be discarded or otherwise replaced or replenished a process that can take even longer than the separation. This method is also subject to resolution problems due to the different mobility's imparted by different fluorescent tags. Since each different tag affects the mobility differently, the movement of the tagged molecules through the gel is not purely dependant on the size of the original DNA and will be affected by which tag has been incorporated.

Methods that use electrophoresis for high throughput sequencing are slow, complex, and expensive and the equipment requires constant maintenance. The equipment must be reconditioned between every run costing time and additional consumables. In order to sequence a single organism in a reasonable time frame it is necessary to perform a very high volume of reads in a short period. Since electrophoresis is slow, many electrophoresis machines must be purchased making the sequencing process very expensive (if not impractical) in both capital costs as well as maintenance costs.

Another approach to sequencing DNA involves the use of mass spectrometers. This method uses the mass spectrometer to determine the sequence from mass measurements made on copies of the original sequence or on probe molecules.

U.S. Pat. No. 5,003,059 Brennan discloses a nucleic acid sequencing method using mass tags that are substituted for the corresponding base. This method uses gel electrophoresis to separate individual nucleic acid sequences prepared by the chain termination method. Each of the terminating bases contain a unique isotope that can be detected using a mass spectrometer. As the nucleic acid sequences exit the electrophoresis medium, they are combusted and run through a mass spectrometer. While measurements of the mass of the molecules exiting the chromatograph are fast, the electrophoresis limits the speed of this method.

U.S. Pat. No. 5,643,798 Beavis et. al. teaches a method of sequencing using matrix assisted laser desorption/Ionization time of flight mass spectrometry. The analysis is performed on nucleic acid fragments of different lengths prepared using the either the Maxam and Gilbert method or the Sanger and Coulson method. The sequence of the original nucleic acid is determined by measuring the mass of each of the complimentary nucleic acid fragments. The base at each position is deduced by comparing the mass differences. The sequence can then be inferred from these differences. The preferred method taught by Beavis performs the sequencing on four separately prepared collections of nucleotide fragments: one each for fragments terminating in A, G, C and T. Beavis mentions that measuring each collection separately instead of as a mixture, is preferred since both the mass resolution and accuracy of the mass spectrometer must be much greater to be reliable enough to accurately determine the sequence. The method that Beavis teaches is an improvement over slower methods incorporating electrophoresis, however it is very dependant upon the resolution of the mass spectrometer to make an accurate determination of the sequence.

U.S. Pat. No. 5,691,141 Koster discloses a nucleic acid sequencing method using a mass spectrometer to measure the mass of fragments of nucleic acid fragments also produced using the Sanger Sequencing strategy. In this case, each fragment has incorporated a base specific, mass-modified chain terminating nucleotide. As in Beavis's method, the specific base at each position is determined by the difference in mass between each of the fragments, however Koster teaches that by using mass-modified nucleotides, the ability to resolve different bases is improved. Koster also teaches that by using mass-modified nucleotides more than one sequence can be measured at once allowing simultaneous sequencing. This method improves the possible throughput since it provides for sequencing more than one sequence at once, however it is still very dependent upon the resolution of the mass spectrometer to accurately determine the sequence.

A common limitation that time of flight mass spectrometers have is the resolution that they are able to achieve when trying to differentiate between large molecules with slight differences in mass. As the total mass of the sequence increases it becomes increasingly difficult to resolve the mass differences necessary to accurately identify the base for a given position. To achieve good resolution, molecules of like size must be tightly clumped with very little overlap to provide discrete arrival times at the detector. The clumps can then be resolved to detectable, discrete peaks between different size molecules instead of a continuous output. Since the velocity of the molecule is proportional to its mass, small relative differences in mass result in small differences in velocity. One major source of error is due to initial velocities that the molecules have before acceleration. These differences in velocity provide error that is difficult to distinguish from velocity differences cause by differences in mass. This means that measurements on molecules that differ by only the slight difference in molecular mass between A, C, G or T become more difficult to resolve as the size of the entire molecule increases. This method has typically been limited to sequencing shorter lengths of nucleic acid due to the accuracy and resolution required for larger molecules.

The detectors in time of flight mass spectrometers are typically less sensitive to larger molecules with low energies. If a mixture of nucleic acid sequence fragments is analyzed that contains a large number of fragments of different lengths, the small molecules will be detected, but the larger molecules must be accelerated at the end of the drift region in order to provide enough impact to provide a signal on the detector. This introduces additional complexity and source for error.

The detectors also have a limited life that depends on the number of molecules that strike them. This means that regular maintenance and replacement is usually required to keep them accurate, this increases cost and down time. This is problematic for a machine that is to be used for high volume sequencing since by the very nature of the process, very large quantities of molecules must be run.

Background noise is also a problem with much of the prior art. Collisions of stray molecules with the detector cause noise that reduces sensitivity. Molecules that either are from the desorption matrix or became fragmented during acceleration and or drift will produce a signal that is not discemable from the actual molecules being measured.

While the mass spectrometer can provide fast reads, numerous practical limitations prevent it from being the high throughput tool that is needed. Therefore, there is a need to be able to determine the sequence of nucleic acids in a much faster and more economical way. Whatever the precise merits, features and advantages of the above cited references, none of them achieves or fulfills the purpose of the present invention as set forth below.

### BRIEF SUMMARY OF THE INVENTION

In one example embodiment, a method for analyzing at least one molecule is provided. The method comprises: providing at least one molecule; isolating the at least one molecule; causing the at least one molecule to emit a signal; and detecting the signal.

Another example embodiment provides a novel device for the analysis of nucleic acid fragments comprising: a source of chromophore or fluorophore tagged nucleic acid fragments, the chromophore of fluorophore being distinguishable by the spectral characteristics; means for vaporization and acceleration of said nucleic acid fragments; means for introducing the tagged nucleic acid fragments to the vaporization and acceleration means; a drift region; said vaporization and acceleration means being located at one end of said drift region and directed so as to propel said nucleic acid fragments through said drift region; detecting means located at the end of said drift region generally opposite said accelerating and vaporization means; said detecting means comprises means for inducing emission from the tagged nucleic acid fragments and means for detecting emissions from said tagged nucleic acid fragments and distinguishing said tagged nucleic acid fragments.

Another example embodiment provides a vaporization and ionization means comprising electro-spray ionization.

Another example embodiment provides a vaporization and ionization means comprising matrix assisted laser desorption ionization.

Another example embodiment comprises a source of illumination comprising a laser.

Another example embodiment comprises a means for detecting emissions comprising a prism and one or more photo detectors located at positions corresponding to unique spectral positions.

Another example embodiment comprises a method of determining the sequence of nucleic acids comprising the following steps:

Introduction of chromophore of fluorophore tagged nucleic acid fragments, said chromophore of fluorophore being distinguishable by its spectral characteristics; vaporization of said nucleic acid fragments; acceleration of said nucleic acid fragments; stimulation of said nucleic acid fragments by external means so as to induce emissions from said tag; and detection of said emissions.

Another example embodiment comprises a device for the determination of the sequence of a nucleic acid sample comprising: a generally tubular chamber; said chamber being evacuated sufficiently to prevent degradation of said sample during analysis; means for electrospray ionization of said sample; an accelerating grid adjacent the injector; an un-obstructed section of sufficient length to allow separation of said sample after acceleration by said accelerating grid; a laser directed to intersect the path of flight of said sample, positioned at the end of said un-obstructed section, opposite said accelerating grid; a photo-detector located sufficiently close to said intersection of said illumination source and said path of flight of said sample.

Another example embodiment comprises a photo-detector located sufficiently close to said intersection of said illumination source and said path of flight of said sample.

Another example embodiment comprises an un-obstructed section of sufficient length to allow separation of said sample after acceleration by said accelerating grid.

Another example embodiment comprises a source of illumination directed to intersect said path of flight of said nucleic acid fragments, positioned at the end of said tubular chamber, opposite said vaporization and acceleration means.

Another example embodiment comprises a chamber being evacuated sufficiently to prevent degradation of said nucleic acid fragments during analysis.

Another example embodiment comprises at one end of said chamber, means for vaporization and acceleration of said nucleic acid fragments along a path of flight generally in the direction of the axis of said tubular chamber.

Another example embodiment provides a method for analyzing at least one molecule Comprising: Providing item to be analyzed; isolating the item to be analyzed; causing the item to be analyzed to emit a signal.

Another example embodiment provides a method for analyzing at least one molecule comprising: providing at least one molecule; isolating the at least one molecule; causing the at least one molecule to emit a signal; and detecting the signal.

Another example embodiment provides a method for analyzing at least one molecule comprising: providing at least one molecule; causing the at least one molecule to have a non-neutral charge; separating the at least one molecule based on its mass to charge ratio; causing the at least one molecule to emit a detectable signal; detecting said signal; recording said signal.

Another example embodiment provides a Method for analyzing at least one molecule comprising: providing at least one molecule; accelerating the at least one molecule; allowing the at; least one molecule to travel a distance; causing the at least one molecule to emit a detectable signal; detecting said signal; recording said signal.

Another example embodiment provides a method for determining the identity of at least one base of at least one polynucleotide comprising:; providing a population of fluorescently labeled fractions; each fraction having a unique fluorescent label characteristic of the base at its end position; accelerating the population of fractions in a manner so as to impart generally the same amount of energy to each molecule; allowing the population of fractions to travel a distance sufficient to separate like fractions into differentiable groups; causing at least one of the fluorescent labels on at least one of the fractions to fluoresce; and detecting the signal emitted from the label.

Another example embodiment provides a method of sequencing a group of molecules, wherein each molecule comprises multiple sub-units of differing sub-unit types, wherein each of the molecules includes at least one tag specific to the sub-unit type, the method comprising: accelerating said molecules, separating said molecules dependant upon at least said accelerating, and radiant detecting of each of the at least one tags by the tag type of each of the at least one tags.

Another example embodiment provides radiant detecting comprises electromagnetic radiant detecting.

Another example embodiment provides radiant detecting comprising phosphorescent radiant detecting.

Another example embodiment provides radiant detecting comprising fluorescent radiant detecting.

Another example embodiment provides radiant detecting comprising thermal radiant detecting.

Another example embodiment provides radiant detecting comprising radioactive radiant detecting.

Another example embodiment provides radiant detecting comprising particle radiant detecting.

Another example embodiment provides radiant detecting comprising chemical-reactive radiant detecting.

Another example embodiment provides radiant detecting comprising detecting the radiation of the tag with a detector.

Another example embodiment provides radiant detecting comprising electromagnetic radiant detecting.

Another example embodiment provides radiant detecting comprising phosphorescent radiant detecting.

Another example embodiment provides radiant detecting comprising fluorescent radiant detecting.

Another example embodiment provides radiant detecting comprising thermal radiant detecting.

Another example embodiment provides radiant detecting comprising radioactive radiant detecting.

Another example embodiment provides radiant detecting comprising particle radiant detecting.

Another example embodiment provides radiant detecting comprising chemical-reactive radiant detecting.

Another example embodiment provides radiant detecting comprising detecting the radiation of a detection substance upon contact with the tag.

Another example embodiment provides radiant detecting comprising electromagnetic radiant detecting.

Another example embodiment provides radiant detecting comprising phosphorescent radiant detecting.

Another example embodiment provides radiant detecting comprising fluorescent radiant detecting.

Another example embodiment provides radiant detecting comprising thermal radiant detecting.

Another example embodiment provides radiant detecting comprising radioactive radiant detecting.

Another example embodiment provides radiant detecting comprising particle radiant detecting.

Another example embodiment provides radiant detecting comprising chemical-reactive radiant detecting.

The p In molecular biology and materials science there is a growing need for the identification and characterization molecules. The device of the current invention would allow the determination of various characteristics such as mass, absorbance and fluorescence signatures and possibly molecular structure.

An embodiment of the invention is an apparatus for determining the sequence of DNA molecules, however the invention can be applied to many analytical purposes in characterizing molecules. A prototype generic claim for this device and method could be:

A method for analyzing at least one molecule comprising: accelerating the at least one molecule; allowing the molecule to travel a distance; remotely detecting a signal from the molecule after traveling said distance; recording said signal from said detecting.

The apparatus for determining the sequence of DNA is similar to a time of flight mass spectrometer and has four basic components:
1. A molecule accelerator that ionizes and accelerates the molecule of interest. This can be an apparatus such as an electro-spray device or a matrix assisted laser desorption ionization device.
2. A flight tube that is connected to the accelerator and provides a path for the molecules to travel after they are accelerated. This flight tube would be held at a vacuum to minimize collisions during the flight of the molecule being analyzed.
3. A detection device that comprises:
a laser directed generally normal to the flight path of the molecules and located at the end of the flight tube opposite from the accelerator;
4 photon detectors such as photo-multiplier tubes located in the same plane as the laser and oriented generally normal to the laser beam;
a refractor for dispersing light into its component colors and directing the light at one of each of the 4 photon detectors.
4. A data recording device that records the signals from each of the detectors.

The operation of the apparatus is as follows: The DNA to be analyzed is prepared in a manner typical for analysis in a 4 color capillary sequencing device. This process produces a population of molecules that range in length from a few molecules to the original length of the DNA molecule to be analyzed. During the sequencing reaction a fluorescent tag is incorporated at the end of each of these molecules. The tags fluoresce when excited by a laser and emit one of 4 colors representing the base for that end position.

The DNA prepared as described above is introduced into the accelerator component of the apparatus of the current embodiment of the invention. A group of these molecules are ionized and accelerated by the accelerator and directed to travel down the flight tube.

As a result of traveling the distance of the flight tube the molecules are fractionated by length. Since all molecules are imparted the same amount of energy by the accelerator, each molecule of a given length travels at a different velocity. The smallest molecules travel the fastest and the next smallest next fastest, etc. until the largest molecules which travel the slowest. This velocity difference causes the molecules to pass the detector at different times and thus accomplishes the fractionation.

As each molecule group passes the detector they are illuminated by the laser. This illumination causes the fluorescent tags to emit light which passes through the refractor and is directed to the appropriate photo detector.

The data recording device records the detector signal strength and the time detected.

After all of the molecules have passed the detector, the data recorded then can be analyzed and the exact sequence of the original DNA molecule determined by correlating the wavelength detected and the order in which it was detected.

The present invention is shown as a block diagram in FIG. 1. The present invention comprises a sample accelerator 1, a drift tube 2 and a detector 3. The chamber in the drift tube 8 and the area inside the detector are maintained at high vacuum by vacuum pumps connected at ports 5 and 6. The sample accelerator vaporizes, ionizes and accelerates the sample molecules down the drift tube along the path 7 and through the detector chamber 15. While passing through the detector 3, the sample ions are illuminated by the laser beam 11 causing the fluorescent dye terminator molecules incorporated into the sample molecules to emit light. The photo detector 9 then detects this light. The particular dye terminator incorporated at the end of the molecule corresponds to the original nucleotide that. Once paste the detector, the sample molecules are then cleared from the chamber mainly by the vacuum pump connected to port 6.

Referring to the block diagram in FIG. 1, the sample molecules to be analyzed are vaporized and ionized by ionizing means 1. The ionizing means 1 can be any device that provides a source of ionized molecules of sample without causing excessive degradation of the sample molecules. Devices that are commonly used to do this use techniques such as Matrix Assisted Laser Desorption Ionization (MALDI) and Electrospray Ionization (ES). These techniques are commonly used to provide sample ion sources for Time of Flight Mass Spectrometers and are well known. Each device has particular advantages and disadvantages but serves as means to convert the sample to be analyzed to a gaseous ionized collection of molecules. Once the sample molecules leave the ionizing means 1. The ionizing means accelerates the sample molecules to a velocity that is proportional to their mass to charge ratio. Thus, the smaller molecules will have higher velocities than the larger molecules. The molecules exit the ionizing means 1 through exit port 14 with a velocity directed down the drift tube 2. The dashed line 7 represents the flight path of the molecules, which travel down the drift tube past the detection point 13. As the molecules travel the distance down the drift tube, the smaller (faster moving) molecules travel the distance faster than the larger molecules. This results in a separation of the sample such that the molecules pass the detection point in order of increasing size with smallest arriving first and largest arriving last. The chamber areas in the drift tube 7 and detector 15 are maintained at a high vacuum. The vacuum should be sufficient so as to prevent collisions between the sample and stray molecules causing excessive fragmentation and disruption of the sorting process.

The sample to be sequenced is injected at 1. Very quickly after injection the sample breaks into very small droplets that evaporate and leave the individual molecules in a charged state.

After the sample is fully vaporized the accelerating grid 2 is turned on accelerating the molecules from the sample through the grid. After passing through the grid they travel down a drift section that is an un-obstructed section of the chamber. This section is of sufficient length to allow separation of said sample after acceleration by the accelerating grid. The molecules are accelerated to a velocity that is proportional to their mass to charge ratio. Therefore molecules of like mass (size) will be accelerated to very near the same velocity. As the molecules travel down the drift section, the fastest (smallest) molecules are the first to reach the detector section. The next smallest molecules arrive next and so on until all of the molecules from the sample have passed the detector section.

An object of the invention is to make large-scale sequencing of nucleic acids faster, simpler and lower in cost. Several other objects and advantages of the present invention are to provide a method and an apparatus to sequence polymeric or chain type molecules such as nucleic acids:
a) in larger volumes in a shorter amount of time;
b) having larger molecular size with greater accuracy;
c) as a continuous process without requiring reconditioning between each run;
d) with lower maintenance requirements;
e) with a lower sequencing cost per base.

An example embodiment of the invention is a method and apparatus for determining the sequence polymeric or chain type molecules such as nucleic acids. This example embodiment comprises a source of chromophore or fluorophore tagged molecule fragments each being distinguishable by its spectral characteristics; a means for vaporization and acceleration of the molecule fragments; means for introducing the tagged molecule fragments to the vaporization and acceleration means; a drift region having the vaporization and acceleration means located at one end of the drift region and directed so that it propels the molecule fragments through the drift region; detecting means located at the end of the drift region generally opposite the accelerating and vaporization means. The detecting means comprises means for inducing emission from the tagged molecule fragments; means for detecting emissions from the tagged molecule fragments and distinguishing the tagged molecule fragments.

Sequencing of polymeric or chain type molecules such as DNA is accomplished by producing duplicate copies of varying lengths of the original sequence that are terminated with a base specific chromophore or fluorophore. Four different chromophores or fluorophores are used (one for each possible nucleotide) and each terminating molecule emits a unique emission spectrum when excited. The prepared DNA or nucleic acid is then loaded into the present invention for analysis. The nucleic acid fragments are then vaporized, ionized and accelerated by an electric field and directed down the drift region. The nucleic acid fragments are all subjected to approximately the same force in the accelerating field; however, since each fragment of a different length has a different mass, each is accelerated to a different final velocity. As the nucleic acid fragments travel through the drift region, their differences in velocity cause them to be sorted from smallest to largest, the smallest arriving first and largest last. The detector illuminates the molecules as they pass and a sensor receives the resulting emission. The detector is designed to sense characteristic emission spectrum of each tagged nucleotide allowing determination of the individual bases. The output from each sensor is then an accurate, ordered sequential representation of the bases in the original molecule under analysis.

This design achieves very high throughputs in contrast with electrophoresis. Electrophoresis can typically take at least an hour for the sample to pass completely by the detector compared to fractions of a second for the present invention. The present invention requires virtually no reconditioning. All that is necessary to prepare the machine to sequence another sample is for the vacuum pump to clear the molecules from the previous sample out of the vacuum chamber, which happens very quickly.

The present invention has advantages over mass spectrography since the detection method depends on detection of the emission from florescent tags not precise measurements of time between discrete collisions.

The apparatus required is relatively simple with very few parts to fail; therefore, the maintenance requirements are lower than the prior art. The machine can be made to operate automatically and there is next to no reconditioning required between runs so the labor cost per sample is lower than the prior art.

Other and further objects, advantages and features of the present invention will become apparent from a consideration of the following discussions and drawings describing various embodiments of the invention.

### BRIEF DESCRIPTION OF THE SEVERAL VIEWS OF THE DRAWING

FIG. 1 shows a schematic diagram of a example nucleotide-sequencing device in accordance with the current invention.

FIG. 2 shows a symbolic representation of a hypothetical nucleic acid sequence paired with a complimentary nucleic acid copy terminated with a base specific tag molecule.

FIG. 3 shows a symbolic representation of a vaporized group of hypothetical nucleic acid copies of different lengths; illustrating a random special orientation of the different length molecules.

FIG. 4 shows a symbolic representation of the same molecules in FIG. 3 shortly after being accelerated; illustrating separation of the sizes.

FIG. 5 shows a symbolic representation of the same molecules in FIG. 3 after being accelerated and traveling for sufficient time to effect significant separation by size.

FIG. 6 shows a schematic representation of the detector optics of an example embodiment.

FIG. 7 shows a symbolic representation of a group of molecules under analysis and the corresponding outputs from the detectors sensing them.

FIG. 8 shows a cross section of an example detector having a single photo detector.

### DETAILED DESCRIPTION OF THE INVENTION

An example embodiment of the invention is an apparatus for determining the sequence of bases in a nucleic acid such as DNA or RNA. The basic steps involved in the process include:
a) Making copies ranging in length from 1 nucleotide to the same length as the molecule under analysis
b) Incorporating a base specific molecule at the end of the copy that corresponds to the base of the original molecule at that position and has a tag molecule that emits a uniquely identifiable spectrum when induced by external means
c) Vaporizing the molecules
d) Accelerating the molecules in a way so as to impart substantially the same energy to each molecule
e) Allowing the molecules to travel for a sufficient time after acceleration so that the molecules are able to separated as a consequence of their differences in velocity
f) Inducing emission from the molecules in a localized area of the path of travel after time for separation has elapsed
g) Detecting the emissions from the molecules

A detailed description of each of the steps listed above will now be given generally in the order that they are presented.

The nucleic acid that is to be analyzed is prepared by producing copies ranging in length from a few nucleotides up the same length as the original sample molecule. When these copies are produced care is taken so as to produce generally equivalent numbers of molecules of each given length. At the end of each molecule, a fluorescent tag is incorporated in place of the original nucleotide. Four different tags are used in the preparation of the copies, one for each of the four possible nucleotides. Each of these tags has unique emission spectra when induced by external means such as illumination by a light source such as a laser.

There are various techniques for preparing the samples to achieve the desired results mentioned above. The most common method involves the use of the enzymatic chain termination reaction. This method is widely used and well known. This technique involves the Polymerase Chain Reaction (PCR) to make copies of the original sequence. During the copying, a dideoxynucleoside triphosphate with a fluorescent tag molecule attached is incorporated randomly during PCR this halts the copying of the chain at the point where it is incorporated. Sufficient PCR cycles are run so that large enough populations of base specific terminated fragments of different lengths exist to allow detection by the detector as described later in this disclosure. This process is generally referred to as a sequencing reaction. This method of preparation is commonly used in preparing molecules for sequencing using electrophoresis. Several variations of this technique exist, are well known and are mostly based on methods proposed by Sanger, F., Nicken, S. and Coulson, A. R. Proc. Natl. Acad. Sci. USA 74, 5463 (1977) and the methods proposed by Maxam, A. M. and Gilbert, W. Methods in Enzymology 65, 499-599 (1980).

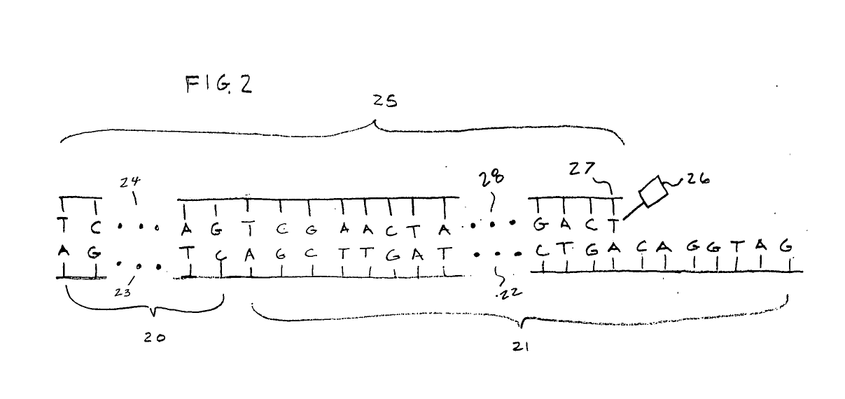

FIG. 2 shows a schematic view of a short strand of DNA prepared using a sequencing reaction. 21 represents the original sequence of nucleotides that is to be analyzed. The ellipses 22,23,24 and 28 indicate the positions of an arbitrary number of intervening bases that are not shown due to space limitations in the drawing. The bases shown in this view are A representing adenine, C representing cytosine, G representing guanine and T representing thymine. The particular sequence shown has no particular significance and was chosen randomly for the purposes of illustration only. The invention does not depend upon any specific bases or number of bases in the molecule under analysis. 20 represents the primer region. The strand shown generally at 25 above and complementary to the original sequence represents the copy of the original sequence generated by PCR. The molecule is shown in the state after the polymerase has completed the copying of the original sequence 21 and the polymerization has been terminated by molecule 27. The terminating molecule 27 has tag 26 attached to it. In the case shown, the terminating molecule is shown as a T and is complimentary to the corresponding molecule A on the original sequence.

In the example embodiment, the Terminating molecule 27 that is incorporated is a dideoxynucleoside triphosphate with a fluorophore molecule 26 attached to it. The terminating molecule 27 is shown as a T in this case since T is complimentary to A, this was chosen for illustration. What is important is that the molecule is complimentary to the base on the original sequence for that position. The tag molecule 26 in this case is a fluorophore. It emits light when stimulated by an external source such as a laser. The emission spectrum of this molecule is chosen to be unique for the particular terminating molecule that it is attached to. For example the terminating molecule that is complementary to A will have a unique fluorophore that will have a unique emission spectra from the fluorophore that is attached to the terminating molecule complimentary to G and likewise unique for C and T. This allows each terminating molecule to be uniquely identified when stimulated so that they can be differentiated from the other bases. The tag molecule 26 could alternatively be a chromophore or any molecule that will emit a detectable emission when stimulate by an external source and that can be uniquely distinguished from the emissions of the other tag molecules in the sample. The present discussion refers to the analysis of DNA and the bases present therein, however, RNA could be analyzed in a similar fashion. In the case of RNA, it would be necessary to use a terminating molecule that would be complimentary to Uracil and use a polymerase appropriate for the reaction. The present invention is not intended to be limited only to the sequencing of DNA.

During the sequencing reaction, a sufficient number of copies of the original sequence are generated to provide sufficient signal for the detector when stimulated. As the molecules are synthesized by the polymerase, the terminating molecules are randomly incorporated which halts extension. The reaction is prepared to produce a generally uniform quantity of copies ranging from the first base to the entire length of the original molecule.

The Example sequencing reaction for the present invention makes uses of the polymerase chain termination reaction however; any method that yields copies of the original sequence that can be distinguished from the other terminating molecules representing a different base is acceptable. What is important for the process is to have one or more copies of the original sequence for each base in the original sequence and that each copy has a length representative of the position that each base occupies. For example if a molecule having 5 bases were to be analyzed there should be at least 5 molecules with lengths of 1, 2, 3, 4 and 5 nucleotides. Each of the 5 molecules will have a terminating molecule that is complimentary to the original base at the terminating position in the original molecule. The terminating position refers to the position of the base at the location where copying was terminated.

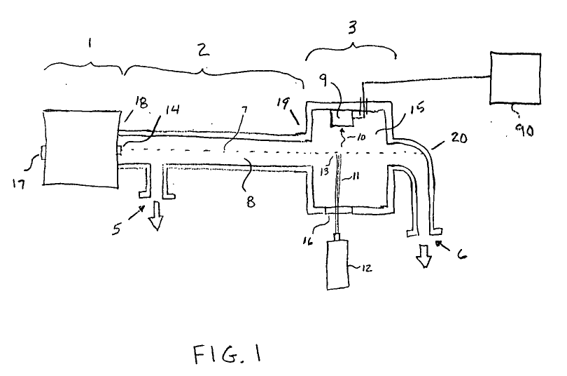

Once the sample has been prepared as described above it is loaded into the apparatus of the present invention shown generally in FIG. 1. The example embodiment of the present invention comprises a source of nucleic acid fragments each being distinguishable by its spectral characteristics as described above; a means for vaporization and acceleration of the nucleic acid fragments shown generally at 1; means 17 for introducing the nucleic acid fragments to the vaporization and acceleration means; a drift region 2 having two ends 18 and 19 and having the vaporization and acceleration means 1 located at one end 18 of the drift region and directed so that it propels the nucleic acid fragments through the drift region along the path generally represented by the dashed line 7; detecting means shown generally at 3 located at the end 19 of the drift region 2 generally opposite the accelerating and vaporization means 1. The detecting means 3 comprises means 12 for inducing emission from the nucleic acid fragments represented by the dashed line 7; and means 9 for detecting emissions from the tagged nucleic acid fragments, represented schematically by the wavy arrow 10 and distinguishing the tagged nucleic acid fragments.

Referring again to FIG. 1, the vaporizing and accelerating means 1 in the example embodiment is an electrospray device. The purpose of this device is to vaporize the molecules of the sample and accelerate them to a velocity that is proportional to their masses. Typically with electrospray the molecules of the sample are vaporized, ionized and accelerated by an ion accelerator. The velocity that the molecules are accelerated to is proportional to their mass to charge ratio. Electrospray is a common technique used in mass spectrography for vaporizing and accelerating a sample to be analyzed and is well understood. U. S. Pat. No. 5,015,845 Allen et al shows such a device. This patent is sited for reference; there are many different designs for this technique that will work well for the purposes of the present invention. Electrospray is used in the example embodiment because it accelerates large molecules without causing significant degradation of the molecules and because it lends itself to a continuous process. With electrospray, the sample can be introduced continuously to the device while maintaining the vacuum in the drift region. This means that the drift region 2 and detector 3 do not have to undergo periodic pump downs just to introduce more samples. This is highly desirable in achieving high throughput since it eliminates the down time that would be incurred if these chambers had to be pumped down periodically.

Vaporization and acceleration of the sample may be accomplished by many other methods. Other methods used for mass spectrography may be used providing different advantages as can be appreciated by those skilled in the art. Some of these methods are Matrix Assisted Laser Desorption Ionization, Fast-atom bombardment, Electron impact, Field ionization, Plasma-desorption ionization or Laser ionization. The particular technique is not important as long as the sample is vaporized so that the molecules are generally separated from each other and that the molecules all receive generally the same amount of energy during acceleration. Another important characteristic of the vaporization and acceleration means 1 is that vaporization and acceleration be accomplished without significant degradation of the sample molecules. Significant degradation of the sample for example, would be a situation in which the sample molecules were broken apart to a degree that prevented an accurate signal to be detected by the detection means 3. In this situation, the molecules would not be of the correct size to represent the position of the base nucleotide indicated by the attached tag. The molecule would then be accelerated to a velocity inappropriate for the base. Upon reaching the detector, they would contribute noise that would inhibit accurate determination of the base for that position. If the noise signal from the degraded molecules is greater than the proper signal, it would cause inaccurate detection.

Referring again to FIG. 1, each molecule in the sample is accelerated and allowed to travel down drift region 2 generally along the path indicated by dashed line 7. The drift region 2 has an chamber area 8 which is generally free of obstruction that would inhibit free travel of the molecules. The chamber 8 is maintained at sufficient vacuum so as not to cause collisions with stray molecules that might cause degradation of the sample molecules or significantly disturb the flight of the sample molecules. A vacuum port is shown generally at 5 and is connected to a vacuum pump capable of maintaining sufficient vacuum as described above. The location of this port is shown generally close to the exit port 14 of the vaporizing and accelerating means. This is to more efficiently remove stray molecules entering the chamber 8 through exit port 14. The sample molecules will be essentially unaffected. Alternatively, one or more vacuum pumps may be used and positioned anywhere along the drift region as long as they are capable of maintaining sufficient vacuum as described earlier.

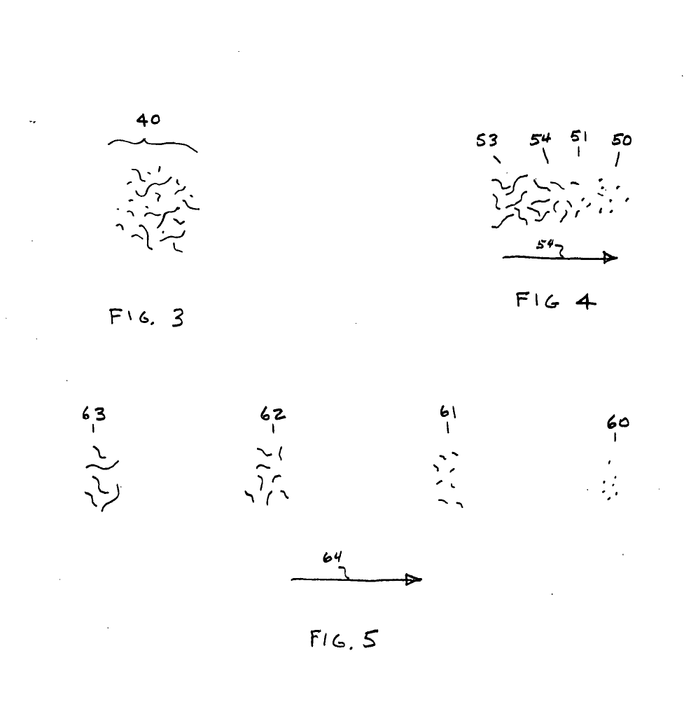

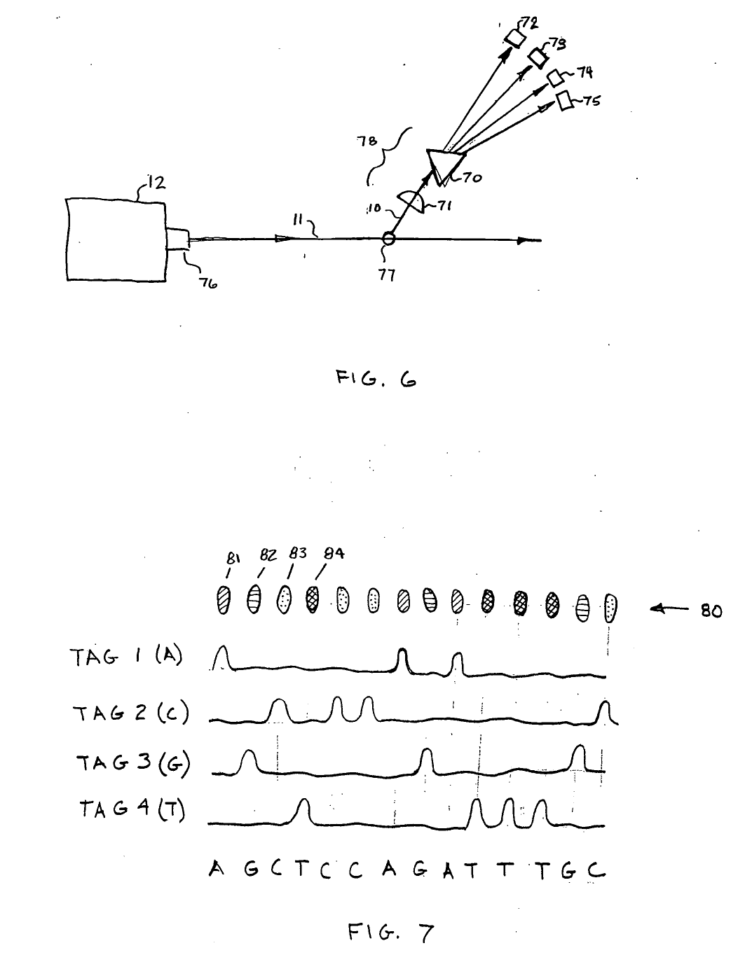

As the sample molecules travel down the drift region 2, the smaller (faster moving nucleic acid fragments) move ahead of the larger ones and are thereby sorted sequentially by size. FIG. 3 shows a hypothetical mixture of sample fragments generally at 40. The mixture is depicted symbolically to represent a mixture of randomly positioned fragments of different lengths. This is representative of the molecules after vaporization and immediately before acceleration. FIG. 4 shows the same molecules as depicted in FIG. 3 shortly after acceleration generally at 50,51, 52 and 53. FIG. 4 illustrates symbolically the process of separation that occurs due to differing velocities of each different fragment length. The arrow 54 shows the general direction of travel of all of the molecules in the sample. The smallest molecules shown generally at 50 have begun to move ahead of the larger molecules shown generally at 51, 52 and 53. The same is true of the next smallest molecules 51, which are shown moving ahead of larger molecules at 52 and 53. Likewise, the molecules at 52 have begun to move ahead of the larger molecules at 53. FIG. 5 illustrates symbolically the same molecules depicted in FIGS. 3 and 4 but at a point in time sufficiently later to allow more complete separation of the molecules. The arrow 64 represents the general direction of travel of the molecules and each different size molecule is represented generally at 60,61,62 and 63 where the smallest molecules are depicted at 60, next largest at 61, next largest at 62 and largest at 63. At this point in time the differences in velocity of each different size molecule has caused a separation and sorting by size to occur. In reality the number of different sized molecules in the sample will usually be more than four as shown in FIGS. 3, 4 and 5; however it can be appreciated that for the purposes of illustration, this small number was chosen to more simply illustrate the separation process in a symbolic manner.

The length of the drift region 2 as shown in FIG. 1, is chosen to allow sufficient distance and time for the molecules to separate sufficiently to allow individual detection of each size molecule. The length of the drift region in the example embodiment is typically 1 to 2 meters but can be longer or shorter depending upon the velocity of the molecules and upon the type of molecule being analyzed. What is important is that the length be sufficient to allow sufficient separation of the molecules for accurate detection by the detector 3.

Referring again to FIG. 1, once the molecules reach the end of the drift region 19, they enter the detector 3. The detector of the example embodiment includes a vacuum chamber 15 that is generally contiguous with the chamber 8 of the drift region and a vacuum pump connected to port 6. The vacuum port 6 has a generally curved section 20 where the sample molecules strike after leaving the detector. The curvature of the port at 20 helps slow down the molecules and deflect them to the vacuum pump connected at 6.

The detector 3 also includes means for inducing emission from the sample nucleic acid fragments, which for the example embodiment is a laser 12. The laser 12 is directed through a transparent window 16 in the wall of the chamber and is aimed to intersect the flight path of the molecules 7 as shown generally at 13. The wavy arrow 10 is a symbolic representation of the emissions from the molecules as they are illuminated by the laser beam 11. In the case of the example embodiment, these emissions are photons. The laser has associated optics that focus and condition the emission inducing photons so that they illuminate the sample molecules in a sufficiently narrow region. The size of the region in the direction of travel of the molecules should be narrow enough to prevent significant illumination of neighboring molecules of different sizes and thus avoid stray signals that could give an erroneous reading. The width of the beam in the plane perpendicular to the flight path of the molecules should be sufficient to illuminate enough of the molecules to generate a detectable signal and maximize the signal to noise ratio. The wavelength of the laser is chosen to best coincide with the excitation maxima for all the fluorescent tag molecules in the sample and thus provide a reasonable compromise for optimal emission from all of the fluorophores.

FIG. 6 shows a block diagram of the optics for a detector in accordance with the present invention. This view is shown looking parallel to a plane that is perpendicular to the flight path of the sample molecules 7 as shown in FIG. 1. Referring to FIG. 6, the laser 12 emits a beam of photons that are that focused and conditioned by optics 76 and is directed to illuminate the sample molecules 77. Some of the photons emitted from the sample are focused and separated into spectral bands by detector optics shown generally at 78. The detector optics shown in FIG. 6 includes a lens 71 and a prism 70. The lens focuses the beam and the prism separates the beam into spectral bands that then strike photomultiplier tubes 72,73,74 and 75.

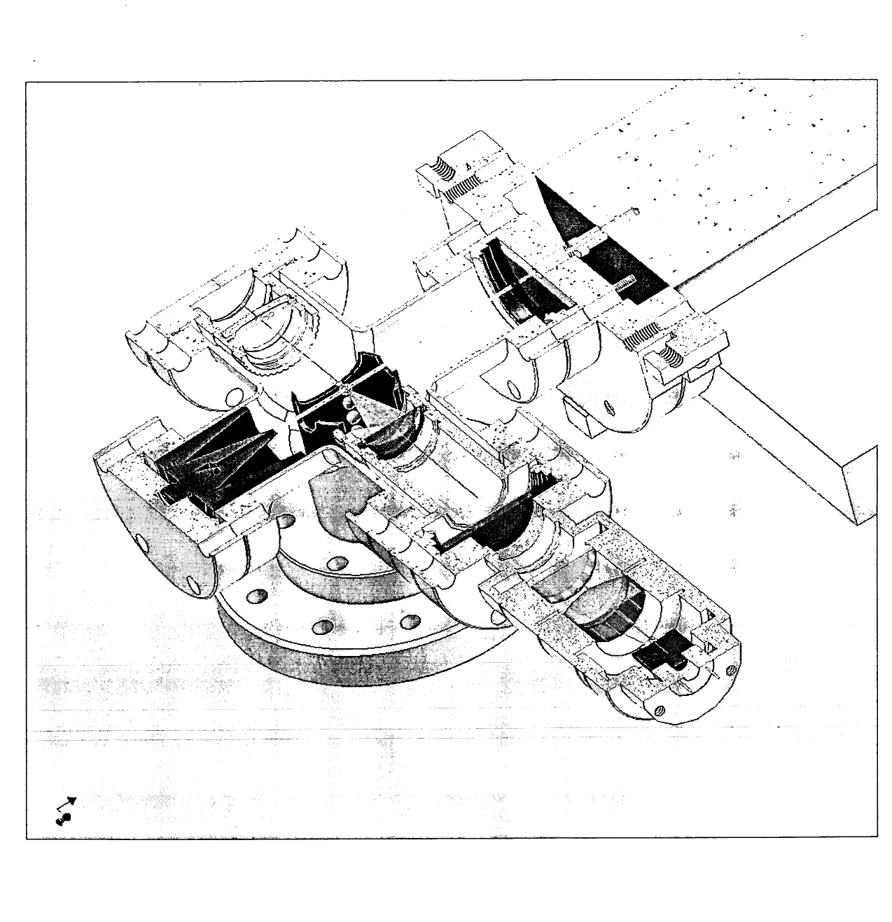

FIG. 7 shows a hypothetical stream of molecules symbolically represented by the ovals generally at 80. Each molecule has a fill pattern that represents the particular tag present in that group of molecules. Group 81 is tagged with the molecule indicating A, group 82 is tagged with the molecule indicating C, group 83 is tagged with the molecule indicating G and group 84 is tagged with the molecule indicating T. Like fill indicates like tags. The lines below the stream labeled Tag 1 (A), Tag 2 (C), Tag 3 (G) and Tag 4 (T) are hypothetical outputs from each of the four detectors 72,73,74 and 75 that correspond to the tags on the molecules shown generally at 80 above. These outputs illustrate amplitude of the output signal vs. time for each detector. As each group of molecules pass through the laser, they are illuminated causing them to fluoresce. The light emitted passes through lens 71 is refracted by prism 70 and directed to one of the four photomultiplier tubes 72 through 75 depending upon the wavelength of light emitted.

The out puts from the photomultiplier tubes are fed into a computer having a high-speed interface to capture the data. As the data comes in from each input, the computer makes the conversion from input source to corresponding base and combines the data sequentially to yield the sequence of the original molecule under analysis. Since the molecules pass the detector in order of increasing size, the order of the out put signals is the same as the order of the original sequence being analyzed.

While for the purposes of disclosure and illustration, the example embodiment has been discussed in detail there are numerous other possible components that can be used in combination to achieve the same purposes and still fall within the scope of the invention. Some of these have been listed above and additional possibilities are listed below for illustration purposes.

An example embodiment of the invention has been explained for sequencing of nucleic acids such as DNA and RNA. Other example embodiments of the invention will be obvious to those skilled in the art and can be used for sequencing proteins or any polymer or chain type molecule. Common elements in the analysis are:
a) the molecules analyzed in the apparatus be duplicates of the original molecule,
b) the duplicates have some distinguishing characteristic representative of the original component molecule occupying the end position,
c) and the distinguishing characteristic be induced to emit some detectable signal that is differentiable from other distinguishing characteristics of the other component molecules being analyzed.

An example detection means for the invention comprises a laser to induce fluorescent emission from the molecules and a photomultiplier to detect these emissions. Other embodiments could use a light from a source such as an electric lamp, directed at the molecules and optical detectors to measure the absorption of light by the molecules. Still another embodiment might sense the emission from molecules tagged with different chromophores. Other embodiments could sense radio frequency emission from molecular tags that emit a distinguishable RF signal when stimulated. Still other embodiments of the detector could sense higher energy emissions such as X-rays when stimulated.

Some alternate methods of stimulation include electron beam, ion beam, and other electro magnetic radiation such as radio frequency, x-ray, ultra violet and gamma ray. High energy collisions with a surface could be used wherein the tag emits radiation of a differentiable spectrum when impact occurs. An example of this is a metal atom incorporated as a tag, and stimulation by a high-energy collision with a surface. What is important to fulfill the purpose of the invention is that the molecules being analyzed emit a distinguishable emission when stimulated.

The example embodiment runs 4 differently tagged molecule groups simultaneously. The different emissions from the different tags distinguish between A, C, G and T. Alternately, a single tagged molecule group could be run and the output data could then be combined afterwards to achieve the same results as running 4 simultaneously. Likewise, any combination of tagged molecule groups could be run together to obtain data for the molecules represented by the tags.

The invention is well suited to fulfill the objects of the invention. Since the molecules to be analyzed are accelerated to a high velocity to effect separation, the travel time through the apparatus is very short, on the order of 10⁻⁶ seconds. Therefore, the time to analyze a single sample is very small. The samples can be loaded into the vaporizer and accelerator in a way such that the vacuum can be maintained and the next sample can be introduced as soon as the previous sample has fully passed the detector. Once the sample is detected, it enters a scrubbing area where it is deflected and immediately removed by the vacuum pump. This allows almost a continuous flow of samples to be run through the apparatus, which allows for very high throughput.

Unlike a mass spectrometer, the present invention does not rely upon impact type detectors like a micro channel device. This means that the detector life does not degrade as a function of sample molecules being run. This provides for significantly longer detector life, higher throughput and the reduction of down time.

In addition, unlike a mass spectrometer, the sequence determination is not dependant upon very precise measurements of differences in arrival times of the molecules to distinguish between terminating molecules. As molecule size increases the difference in mass between different terminating molecules becomes a very small difference compared to the total mass of the molecule. This makes differentiation much more difficult for larger molecules. Differentiation of the terminating molecule in the present invention is not dependant upon precise measurements in arrival time and therefore is not subject to the problems encountered by mass spectrometry. The present invention is therefore, well suited to determine the sequence of larger molecules with greater accuracy than the prior art.

The present invention is capable of very high throughput, requires less maintenance and can be easily automated. This means that sequencing can be preformed on at a significantly higher rate with fewer machines at s substantially lower cost per base. This makes the invention well suited for large-scale sequencing.

The present invention is well adapted to carry out the objects and attain the ends and advantages mentioned, as well as others inherent therein. While, for the purposes of disclosure there have been shown and described what are considered at present to be the example embodiments of the present invention, it will be appreciated by those skilled in the art that other uses may be resorted to and changes may be made to the details of construction, combination of shapes, size or arrangement of the parts, or other characteristics without departing from the spirit and scope of the invention. It is therefore desired that the invention not be limited to these embodiments, and it is intended that the appended claims cover all such modifications as fall within the true spirit and scope of the invention.

I claim:

1. A method for analyzing at least one molecule comprising:
Providing at least one molecule;
Isolating the at least one molecule;
Causing the at least one molecule to emit a signal; and
Detecting the signal.
